(19) United States
(12) Patent Application Publication
(10) Pub. No.: US 2004/0044329 A1
(43) Pub. Date: Mar. 4, 2004

(54) Catheter for cardiac injection and method for delivery of therapeutic agents to specified tissues

(76) Inventor: Leonard A. Trudell, Warwick, RI (US)

Correspondence Address: GENZYME CORPORATION, LEGAL DEPARTMENT, 15 PLEASANT ST CONNECTOR, FRAMINGHAM, MA, 01701-9322, US

(21) Appl. No.: 10/230,497

(22) Filed: Aug. 29, 2002

(51) Int. Cl. A61M 31/00

(52) U.S. Cl. 604/507; 604/165.02

(57) ABSTRACT

Featured is a system for delivering a fluid or therapeutic agent to target tissue of an organ, such as a heart. Such a delivery system includes a catheter having a distal end portion, a proximal end portion and a lumen extending therebetween and a delivery device movably disposed within the catheter at the distal end portion thereof, the delivery device having a first end portion that is configured and arranged for penetrating the target tissue. The distal end portion of the catheter is also configured and arranged such that when the delivery device is extended beyond the distal end portion of the catheter, a portion of the delivery device that is disposed within the distal end portion of the catheter is put into fluid communication with the lumen, whereby fluid or therapeutic agent being communicated via the lumen is fluidly coupled to the first end portion of the delivery device and thence for delivery to the target tissue.

### FIELD OF INVENTION

The present invention relates to catheters and methods for delivery of one or more therapeutic agents to specified tissues, more particularly to catheters having a retractable delivery tip or member which is suitable for use in injecting therapeutic agents into cardiac tissues and more specifically techniques or methods embodying the use of such catheters.

### BACKGROUND OF THE INVENTION

Techniques have been developed for the local delivery of drugs or other therapeutic agent's to the heart. According to one such technique, an open-chest procedure has been adapted for use in delivering therapeutic agents to the heart. In this procedure, the patient's chest is opened surgically to expose the heart; then, a solution comprising one or more therapeutic agents is delivered to the heart tissue via a syringe to make a number of injections in a grid like pattern. There is described in WO 98/32859, for example, a method of enhancing perfusion of blood to a target tissue using an open-chest procedure.

There is described in U.S. Pat. No. 5,551,427 the use of implantable substrates for local drug delivery at a depth within the heart wall (myocardium). According to this technique, an implantable helical injection needle is screwed into the exterior wall of the heart and connected to an implanted drug reservoir outside the heart. This system allows injection of drugs directly into the wall of the heart acutely by injection from the proximal end, or on an ongoing basis by a proximally located implantable subcutaneous port reservoir, or pumping mechanism. Also described therein are implantable structures having a coating, which releases bioactive agents into the myocardium. There is also described in U.S. Pat. No. 5,447,533 an apparatus for the delivery of a therapeutic agent wherein the delivery element for attachment to the heart wall is an implantable helical member than is screwed into the exterior wall of the heart.

There is described in U.S. Pat. No. 6,067,988 an apparatus and methods related thereto for creating drug-filled pockets within muscle tissue such as heart tissue. As shown in FIG. 1 thereof, the device punches holes in the myocardium from the outside of the heart and fills the holes with particles comprising a drug.

There is described in U.S. Pat. No. 5,261,889 a catheter for use within an endoscope that includes a retractable needle for the injection of a drug into tissues and an irrigation lumen. The catheter is housed within a lumen of the endoscope allowing for direct visualization of target tissue. The irrigation lumen enables the user to displace blood and other obscuring substances such that the endoscope visual path is clear.

There is described in U.S. Pat. No. 6,165,188 an apparatus in which myocardial revascularization is performed percutaneously using a catheter. The catheter disclosed therein has an end region that is directable to a patients endocardium and a cutting head which can sever tissue from the endocardium, e.g., the catheters described therein are suitable for removing tissue from the inside wall of the heart to create holes therein.

There is described U.S. Pat. No. 6,102,887 a catheter system for injecting therapeutic agents into the body. The catheter includes a distensible penetrating element and a distally located expansion coil and a distally located chamber for holding a therapeutic agent. The distally located chamber or a supply line is coupled to the penetrating element so the therapeutic agent or drugs can be injected into the tissue that is being penetrated.

It thus would be desirable to provide a catheter that is suitable for the delivery of therapeutic agents to the interior of a heart or other internal organs of a patient. In particular, it would be desirable to provide a new cardiac catheter, which can be localized to the interior of the heart, e.g., to the ventricle or other heart chamber. It is also preferable that the catheter is capable of localized injection of a therapeutic agent to specified regions and desired depths of the cardiac tissue. It also would be desirable to provide a cardiac procedure using such catheters that is simple to perform and is substantially non-invasive as compared to other cardiac procedures such as open-heart surgery and other catheterization procedures described herein. Such catheters preferably would be simple in construction and less costly than prior art devices and such method would not require highly skilled users to utilize the catheter of the present invention.

### SUMMARY OF THE INVENTION

The present invention in its broadest aspects features a system for delivering a fluid or therapeutic agent to target tissue of an organ, such as a heart, from inside thereof. Such a delivery system includes a catheter having a distal end, a proximal end and a lumen extending therebetween and a delivery device movably disposed within the catheter at the distal end thereof, the delivery device having a first end that is configured and arranged for penetrating the target tissue.

The distal end of the catheter is configured and arranged so the delivery device is in one of a first position where the first end of the delivery device is disposed within the distal end of the catheter or in a second position where the first end of the delivery device is extended beyond the distal end of the catheter. The distal end of the catheter also is configured and arranged such that when the delivery device is in the second position a portion of the delivery device that is disposed within the distal end of the catheter is put into fluid communication with the lumen, whereby fluid or therapeutic agent being communicated via the lumen is fluidly coupled to the first end of the delivery device and thence for delivery to the target tissue. In a particular embodiment, the catheter is configured and arranged such that the distal end thereof can be positioned within the heart of a patient and wherein the delivery device is configured and arranged to deliver the fluid or therapeutic agent to tissues of the patient's heart.

When used to deliver such fluid or therapeutic agent to tissues of the heart, the distal end of the catheter is localized to the interior of the heart through the vasculature with the delivery tip completely retracted into the catheter to prevent accidental damage to the vasculature, the aorta, the aortic valve, or healthy portions of heart tissue. After localizing the distal end of the catheter to the targeted region or target tissues of the interior of the heart, the first end of the delivery device is extended beyond the distal end of the catheter such that a portion of the first end, i.e. a tip or point thereof, penetrates the interior wall of the heart.

According to one aspect of the present invention the delivery device includes a passage that extends lengthwise within the delivery device. A first aperture in the first end of the delivery device is fluidly coupled to the passage and a second aperture in the portion of the delivery device that is disposed within the distal end of the catheter is also fluidly coupled to the passage. With such a configuration, the fluid or therapeutic agent being communicated via the lumen is fluidly coupled to the first end of the delivery device via the first aperture, the passage and the second aperture. In a more particular embodiment, the second aperture of the delivery device is fluidly coupled to the lumen in either of the first or the second positions thereof. In a more specific embodiment, a portion of the passage is configured and arranged so as to essentially form a fluid seal with the delivery device proximal to the first aperture of the delivery device so that fluid or therapeutic agent within the lumen is not discharged from the first aperture of the delivery device when the delivery device is in the first position.

According to another aspect of the present invention, the distal end of the catheter is configured and arranged so as to include a passage in which the delivery device is moved from and between the first and second positions, where a portion of the passage includes a through aperture that is fluidly coupled to the lumen. With such a structure, when the delivery device is moved to the second position, the second aperture of the delivery device is fluidly coupled to the lumen via the through aperture. In more specific embodiments, the distal end of the catheter is configured and arranged such that the second aperture of the delivery device is not fluidly coupled to the lumen when the delivery device is in the first position.

In further embodiments, such a delivery system includes an actuation mechanism having a portion of which that is disposed within the catheter and is operably coupled to the delivery device and/or an adapter located at or near the proximal end of the catheter. The actuation mechanism is particularly configured and arranged so that the actuation mechanism selectively moves the delivery device between, and into, the first position and the second position and the adapter is configured and arranged so as to be capable of being fluidly coupled to the source of fluid or therapeutic agent (e.g., syringe) that is to be communicated via the lumen. In more specific embodiments, a portion of the actuation mechanism is disposed in a passage separate from the lumen or the portion of the actuation mechanism is located within the lumen. An example of a form of the actuation mechanism includes the use of a spring arranged inside another spring wherein rotation of the spring elements relative to each other may cause the distal end portion of the catheter to form a bend and/or to stiffen the catheter and to extend or retract the injection needle or delivery device.

Catheter systems for local delivery of therapeutic agents have many advantages. Approaches for local delivery of therapeutic agents at a depth within a tissue are applicable to the heart and other internal organs and tissues that may be accessed through the vasculature via a catheter system. These catheter systems can deliver drugs to the sites where they are most needed, to reduce the amount of drug required, increase the therapeutic index, and control the time course of agent delivery. This, in turn, improves the viability of the drugs, lowers the amount of drug-drug interactions, lowers the risk to patients, and allows the physician to more precisely control the effects induced. Such local delivery may mimic endogenous modes of release, and address the issues of agent toxicity and short half lives.

Also featured are methods related thereto, including methods for delivering therapeutic agents, drugs or fluids to target tissues of a patient. These perferred delivery methods include inserting the distal end of a delivery catheter into a patient's vasculature; localizing the distal end of the catheter to a specified tissue of an organ through the patient's vasculature; penetrating the specified tissue by extending a portion of a delivery device from the catheter; and delivering a fluid or therapeutic agent to the specified tissue through the catheter and delivery device. In particular embodiments, a fluid or therapeutic agent is injected into the heart by localizing a tip of the delivery device to the interior of the heart, penetrating a specified portion of the interior wall of the heart with the tip and injecting the fluid or therapeutic agent into the tissue.

Other aspects and embodiments of the invention are discussed below.

Definitions

The instant invention is most clearly understood with reference to the following definitions:

As used herein, the term “fluid” shall be understood to have the ordinary meaning thereof and include solutions, suspensions, emulsions and the like which may include cellular extracts or whole cells dispersed therein, viral vectors, plasmids, growth factors, DNA, small molecule pharmaceuticals, peptides and other like biologically active compounds and compositions.

### BRIEF DESCRIPTION OF THE DRAWING

For a fuller understanding of the nature and desired objects of the present invention, reference is made to the following detailed description taken in conjunction with the accompanying drawing figures wherein like reference character denote corresponding parts throughout the several views and wherein:

FIG. 1 is a cross-sectional side view of a delivery apparatus according to one aspect of the present invention;

FIG. 2 is a section view taken along section line 2-2 of FIG. 1;

FIG. 3 is a side view including a partial cross-section of a delivery tip according to the present invention;

FIGS. 4A, B are section views of alternate arrangements for lumens of the catheter of FIG. 1;

FIGS. 5A, B are schematic views of an angled end portion of a catheter according to the present invention with the injection tip retracted (5A) and the injection tip extended (5B);

FIGS. 6A and 6B are cross-sectional side views of a catheter end portion illustrating alternate embodiments for a delivery apparatus according to the present invention with the injection tip retracted (6A) and the injection tip extended (6B);

FIG. 6C is a cross-sectional side view of the catheter end portion illustrating yet another alternate embodiment for a delivery apparatus according to the present invention;

FIG. 7 is a cross-sectional side view of the delivery apparatus of FIG. 6 with the injection tip extended;

FIG. 8 is a cross-sectional side view of the delivery apparatus according to FIG. 6 of the present invention with the injection tip retracted;

FIG. 9 is a section view taken along section line 9-9 of FIG. 7;

FIG. 10 is a side view of a delivery tip of according to another aspect of the present invention;

FIGS. 11A and 11B are side views including a partial cross-section of alternative embodiments of the delivery tip of FIG. 10 delivery tip according to the present invention;

FIG. 12 is an end view of a catheter with the delivery tip of FIG. 11B slidably disposed therein; and

FIGS. 13A-C are side views including a partial cross-section of an alternative embodiment showing a pair of spring members according to the present invention.

### DESCRIPTION OF THE PREFERRED EMBODIMENT

The present invention in its broadest aspects features a delivery system or delivery apparatus that delivers a fluid or therapeutic agent to target tissue of an organ, such as a heart, from the inside of the organ. Such a delivery apparatus or system includes a catheter and a delivery tip movably disposed within the catheter at the distal end thereof, where the delivery tip includes a first end that is configured and arranged for penetrating the target tissue.

The distal end of the catheter is configured and arranged so the delivery tip is in a first position where the first end of the delivery tip is disposed within the distal end of the catheter or in a second position where the first end of the delivery tip is extended beyond the distal end of the catheter. The distal end of the catheter also is configured and arranged such that when the delivery tip is in the second position a portion of the delivery tip that is disposed within the distal end of the catheter is put into fluid communication with a lumen within the catheter. In this way, when the delivery tip is in the second position, fluid or therapeutic agent being communicated via the lumen is fluidly coupled to the first end of the delivery tip and thence for delivery to the target tissue. In a particular embodiment, the catheter is configured and arranged such that the distal end can be positioned within the heart of a patient and wherein the delivery tip is configured and arranged to deliver the fluid or therapeutic agent to tissues of the patient's heart. As also herein described, the present invention also features methods for delivering fluids, therapeutic agents and/or drugs to target tissues utilizing such delivery apparatuses.

Now referring to the various figures of the drawing wherein like reference characters refer to like parts, there is shown in FIGS. 1-3 various views of a delivery apparatus 100 or delivery system according to one aspect of the present invention. The delivery apparatus preferably includes a catheter 110, a delivery tip 120 and an actuating mechanism 140 being coupled to the delivery tip. As disclosed herein, the actuating mechanism 140 may take various forms to deliver the desired fluid to the patient. The catheter 110 further includes an inlet port 130 at or near a proximal end 114 thereof. The inlet port 130 is fluidly coupled to a fluid delivery lumen 150 that is disposed within the catheter. Also disposed within the catheter 110 is a passage 160 in which is located a portion of the delivery tip 120 and a portion of the actuating mechanism 140.

As more clearly shown in FIG. 3, the delivery tip 120 includes a tubular member 121 that is sealed at both a distal end 122a and a proximal end 122b thereof so as to form a interior compartment or chamber 124 therein that generally extends lengthwise along the delivery tip 120. The interior compartment 124 is generally defined by the interior surfaces of the tubular member 121 as well as the ends or the structures sealing each of the distal and proximal ends 122a and 122b.

The distal end 122a of the delivery tip 120 is configured and arranged so as to form a structure, such as a needlepoint, that can penetrate the tissue of the targeted region. In particular embodiments, the distal end 122a of the delivery tip 120 is configured and arranged so as create an incision upon tissue penetration. In more specific embodiments, the distal end 122a of the delivery tip 120 includes at least one cutting edge to facilitate formation of the incision, more particularly the distal end of the delivery tip is configured so as to form a structure having a beveled end with one or more cutting edges and at least one opening adjacent thereto or therethrough.

The proximal end 122b of the delivery tip 120 is configured and arranged to provide a structure or structural member to which an end of the connecting member 142 of the actuating mechanism 140 can be operably secured thereto using any of a number of techniques known to those skilled in the art appropriate for the materials comprising the delivery tip proximal end and the actuating mechanism connecting member. Such techniques include, but are not particularly limited to, adhesives, mechanical fixtures such as clamps and bonding techniques such as welding, brazing and soldering. As discussed further below, in an illustrative embodiment, the proximal end 122b of the delivery tip 120 is further configured and arranged in the form of a flange and is arranged or is alternatively constituted by a flange member 128b that is secured to the tubular member 121 to form one end of the interior compartment 124. In general, the proximal end 122b is configured and arranged to withstanding the structural loading imposed thereon by the actuating mechanism 140 as the actuating mechanism causes the delivery tip 120 to be moved back and forth between the first and second positions.

The wall thickness and the materials making up the tubular member 121 also are set so that the circumferential sidewall of the tubular member is not distorted or buckled as the distal end 122a of the delivery tip 120 is being urged against and penetrates the target tissue. The tubular member 121 and the structures making up the distal and proximal ends of the delivery tip are made from any of a number of sterilizable, biocompatible, corrosion resistant material as is known to those skilled in the art. Such biocompatible materials that are suitable for use include, but are not limited to stainless steel, titanium, ceramics such as alumina, biocompatible plastics such as biocompatible polycarbonates, polyethylene, polypropylene, poly(tetrafluoroethylene), i.e., Teflon, biocompatible polyesters, biocompatible polyamides, other biocompatible polymers and mixtures or blends thereof. Similarly, various coating such as silicone, teflon or silver based materials may be applied to the delivery tip and/or catheters.

There is provided in the distal end 122a of the delivery tip at least one through aperture 126a that extends through the end or a sidewall of the tubular member 121 so one end of the through aperture 126a is fluidly coupled with the interior compartment 124. Similarly, in this embodiment, at least one other through aperture 126b is provided in the proximal end 122b of the delivery tip that extends through the sidewall of the tubular member 121 so one end of the through aperture is fluidly coupled to the interior compartment 124. In more particular embodiments, a plurality of through apertures 126a and 126b are provided in at least one of the distal or proximal ends 122a and 122b of the delivery tip. It should be recognized that the through apertures 126a and 122b are not particularly limited to the illustrated embodiments. These through apertures 126a and 122b are generally located in the tubular member 121 such that when the delivery tip 120 is in the second position one or more of the through apertures 126b are fluidly coupled to the lumen and one or more of the other through apertures 126a are disposed within the target tissue.

In more particular embodiments, the interior compartment 124 and the through apertures 126a and 126b in the ends or sidewall of the tubular member 121 near the distal and proximal ends 122a and 122b are configured and arranged so as to have sufficiently large dimensions to prevent damage to the fluid, therapeutic agent, or any particulate material suspended therein as such fluid/material passes through the through apertures 126a and 126b and through the interior compartment 124. In addition, the cross-section of the delivery tip 120 is preferably configured and sized so that the delivery tip 120 and the catheter 110 can be inserted through the vasculature of a patient and may be of various conventional sizes such as in the range of 5 french to 8 french.

Although a circular cross-section is illustrated for the delivery tip 120, this shall not be construed as a limitation on the particular geometric cross-sectional shape for the delivery tip. It is within the scope of the present invention for the delivery tip 120 to the configured so as to have any of the number of cross-sectional geometric shapes known to those skilled in the art including, but not limited to tapered, circular, oval, elliptical or polygonal cross-sections.

The fluid delivery lumen 150 is configured and arranged so that it generally extends between the distal and proximal ends 112, 114 of the catheter 110. In this embodiment, a long axis of the fluid delivery lumen is generally, substantially parallel to the long axis of the catheter. The cross-sectional shape and size of the fluid delivery lumen 150 is set so as to prevent or minimize damage to the one or more therapeutic agent(s) present in the fluid to be delivered via the fluid delivery lumen to the delivery tip 120. Frequently large therapeutic agents, such as whole cells, cell extracts, DNA and the like can be damaged by laminar flow sheer forces and other mechanical agitations which arise in small cross-sectional conduits. In an illustrative, exemplary embodiment, the fluid delivery lumen 150 has an inner diameter in the range of from about 1.0 mm to about 2.0 mm however, these dimensions are not particularly limiting.

It should be recognized that it is within the scope of the present invention for a portion of the fluid delivery lumen 150 that is remote from the distal end 112 of the catheter to be configured and arranged so the long axis of this portion is oriented parallel or at an angle with respect to the long axis of the catheter. For example, this portion of the fluid delivery lumen 150 can be in the form of a spiral or helix about the catheter long axis as well being made up of segments or sections having different configurations (e.g. alternating straight and accurate/helical sections).

As indicated above, the illustrated embodiment the catheter 110 of FIGS. 1-3 further includes an inlet port 130 at or near a proximal end 114 thereof. The inlet port 130 is fluidly coupled to the fluid delivery lumen 150, where an end of the inlet port is secured to a fluid delivery adapter 132. In more particular embodiments, the fluid delivery lumen 150 can have an open proximal end to which is coupled the adapter 132 or the proximal end of the fluid delivery lumen can be solid and the fluid delivery adapter 132 is coupled to the fluid delivery lumen through a conduit in the sidewall of the fluid delivery lumen near the proximal end of the catheter 110. The fluid delivery adapters 132 that are suitable for use in the present invention are not particularly limited. In particular illustrative embodiments, such fluid delivery adapters 132 include those that accept a fluid from syringes, needles, cannulae, syringe pumps and other fluid delivery mechanisms. In more specific embodiments, such fluid delivery adapters 132 conform to standard luer connections and so as to be coupled to luer type syringes. In the illustrated embodiment, the interior of the catheter 110 proximal to the distal end 112 of the catheter, the fluid delivery lumen 150 and the catheter passage 160 are configured and arranged so as to generally form a chamber 115 within the distal portion of the catheter. This chamber 115 is fluidly coupled to the fluid delivery lumen 150 via a plurality of openings in the distal end of the delivery lumen. In this way, fluid, drugs or therapeutic agents being communicated via the fluid delivery lumen 150 are passed through and into the chamber 115. The catheter chamber 115 has a cross-sectional shape and size that is set so as to prevent or minimize damage to the one or more therapeutic agent(s) present in the fluid to be delivered via the fluid delivery lumen 150 while also preventing inadvertent leakage or delivery of the fluid or therapeutic agent through the distal end of the catheter.

In further embodiments, the delivery tip 120 further includes a first flange 128a disposed between the aperture(s) 126b near the proximal end 122b of the delivery tip and the aperture(s) 126a near the distal end 122a of the delivery tip and a second flange 128b at the proximal end 122b of the delivery tip. The flanges 128a and 128b are configured so as to have an outside diameter generally larger than the cross-section of the tubular member 121 of the delivery tip and such flanges 128a and 128b are substantially perpendicular to a long axis as defined by the distal and proximal ends 122a and 122b of the delivery tip.

In addition, in the illustrated embodiment illustrated in FIGS. 1-3, the outer diameter of the second flange 128b is set so the second flange is slidably disposed with the catheter passage 160, which passage basically constitutes a second lumen preferably having a closed proximal end. The second flange 128b also is sized and configured so as to facilitate aligning the distal end 122a of the delivery tip with the through aperture 113 at the distal end 112 of the catheter. For example, the outer diameter of the second flange 128b is set so as to establish a sliding fit between the outer diameter of the second flange and the inner diameter of the catheter passage 160. In a specific embodiment, the long axis of the catheter passage 160 is generally aligned with the center of the through aperture 113 at the catheter distal end 112.

The first flange 128a is positioned so as to prevent over extension or over retraction of the delivery tip 120. Typically, the first flange 128a is spaced apart from the distal end-wall of the catheter when the delivery tip is in the retracted position and is in contact with the distal end-wall of the catheter chamber 115 when the delivery tip 120 is in the extended position. In a more specific embodiment, the second flange 128b is spaced apart from the distal end of the catheter and is in contact with the distal end-wall of the catheter passage 160 when the delivery tip 120 is in the second position (the extended position). Correspondingly, the first flange 128a is in contact with the proximal end-wall of the catheter chamber 115 and is located proximally of the catheter passage 160 when the delivery tip is in the first position (the retracted position).

The distance between the needle point at the delivery tip distal end 122a and the first flange 128a also establishes the depth of penetration into the target tissue when the delivery tip is in the extended position. It is within the scope of the present invention, to provide a plurality of delivery tips that provide a wide range of distances between the needle point and the first flange 128a. In this way, the delivery apparatus 100 of the present invention can be adapted so as to inject or deliver fluid containing drugs or therapeutic agents into the target tissue at a variety of depths therein. It is also anticipated that a delivery device according to the present invention may be provided wherein the depth of the injection may be adjusted by the user by providing a delivery device with interchangeable delivery tips or adjustable flanges.

The actuation mechanism 140 includes a connecting member 142 coupled to the proximal end 122b of the delivery tip, the second flange 128b and a ring member or actuation member 144 as shown in FIG. 7. The connecting member 142 is preferably formed of flexible, substantially non-compressible materials which are resistant to kinking or deformation upon application of a compressive force formed as a wire, interdigitated springs, woven cables, tubes and other objects that can be housed inside of the catheter passage 160 and through which a mechanical force may be transmitted through at least a segment thereof to the delivery tip 120. Preferably, the connecting member 142 is a sterilizable and/or biocompatible material such as stainless steel, titanium and alloys thereof, Teflon, polyethylene, polycarbonates, polyamides such as Nylon, and other biocompatible biopolymers having a suitable coating thereon.

In this way, the proximal end 122b of the delivery tip 120 is mechanically coupled with or connected to the actuation member 144 that is located at the proximal end of the catheter 110. Actuation of the actuation member 144 applies a force to the connecting member 142 which induces a reversible sliding motion of the delivery tip 120 so that it can move or be shifted between and into the extended and retracted positions. In the retracted position, the first position, a fluid is not dispensed from the delivery tip and the delivery tip is housed within the distal end 112 of the catheter (see for example FIG. 5A). In the extended position, the second position, a portion of the delivery tip including the sharpened penetrating tip and at least one through aperture 126a at the distal end 122a of the delivery tip are extended beyond the distal end 112 of the catheter as is shown in FIG. 1. Further, at least one through aperture 126b at the proximal end 122b of the delivery tip is disposed within the catheter chamber 115 when in the extended position, thereby allowing fluid to be communicated to and out of the at least one through aperture 126a at the distal end 122a of the delivery tip.

In an alternative embodiment, the actuation member 144 applies a rotational force to the connecting member 142. This rotational force is translated to a linear force by a suitable mechanical device such as a spring within a spring, worm gear or the like having at least a segment thereof positioned or located at or near the distal end portion of the catheter 110 so that the delivery tip 120 is slidingly shifted, preferably without rotation, between a retracted and extended position.

As indicated above, a portion of the actuating mechanism 140, the connecting member 142, is disposed with the second lumen or catheter passage 160 and extends from the catheter proximal end 114 to the delivery device proximal end 122b. As also noted above, the catheter passage 160 is sized so that the second flange 128b or the delivery device proximal end 122b is slidably disposed within the catheter passage. In addition, the catheter passage 160 is sized so that the connecting member 142 can move in the intended fashion, for example linearly, within the passage so as to shift the delivery tip 120 between and into the first and second (i.e., retracted and extended) positions.

Although the catheter passage 160 is illustrated as having a generally constant cross-section, this shall not be construed as a limitation. It is within the scope of the present invention for the catheter passage 160 to be configured and arranged so as to have any of a number of configurations, sizes and cross-sections and geometric shapes as is known to those skilled in the art and otherwise appropriate for the intended use. For example, the portion of the catheter passage 160 that slidably receives the delivery tip 120 can be configured and arranged so as to complement the delivery tip and the other portion of the catheter passage housing the connecting member 142 can be configured and arranged so as to complement the connecting member. For example, the catheter passage that receives the delivery tip may include a gradually curved portion (Not shown) that complements a curved delivery tip.

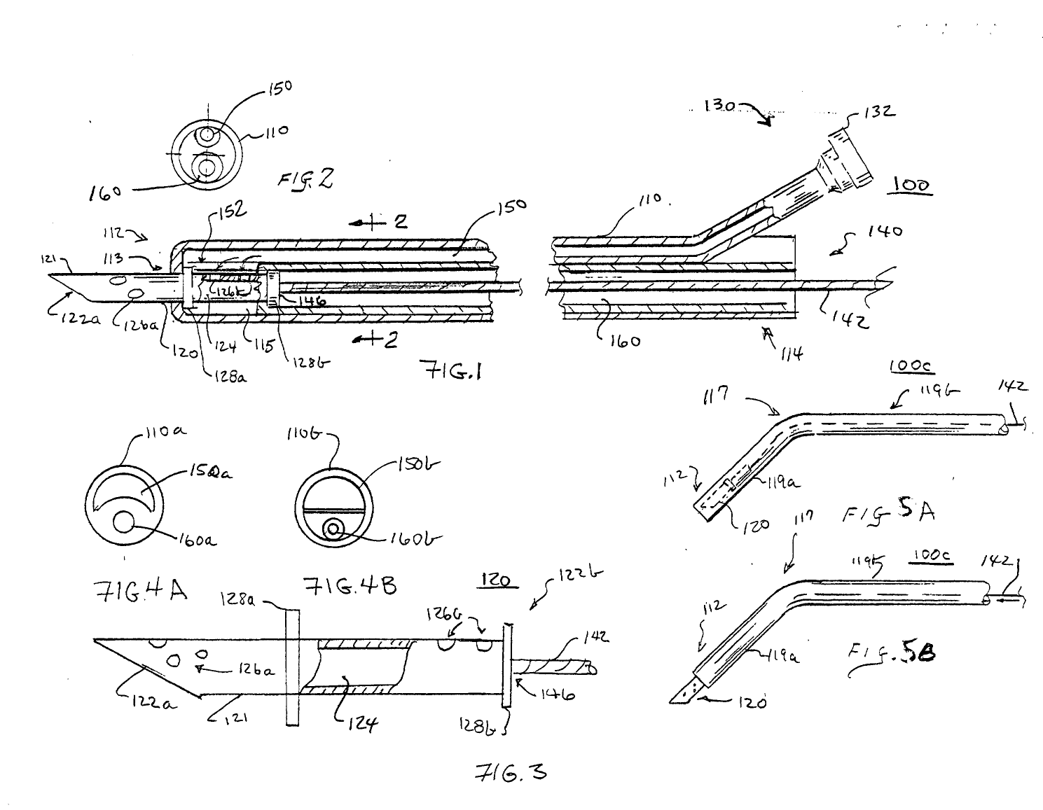

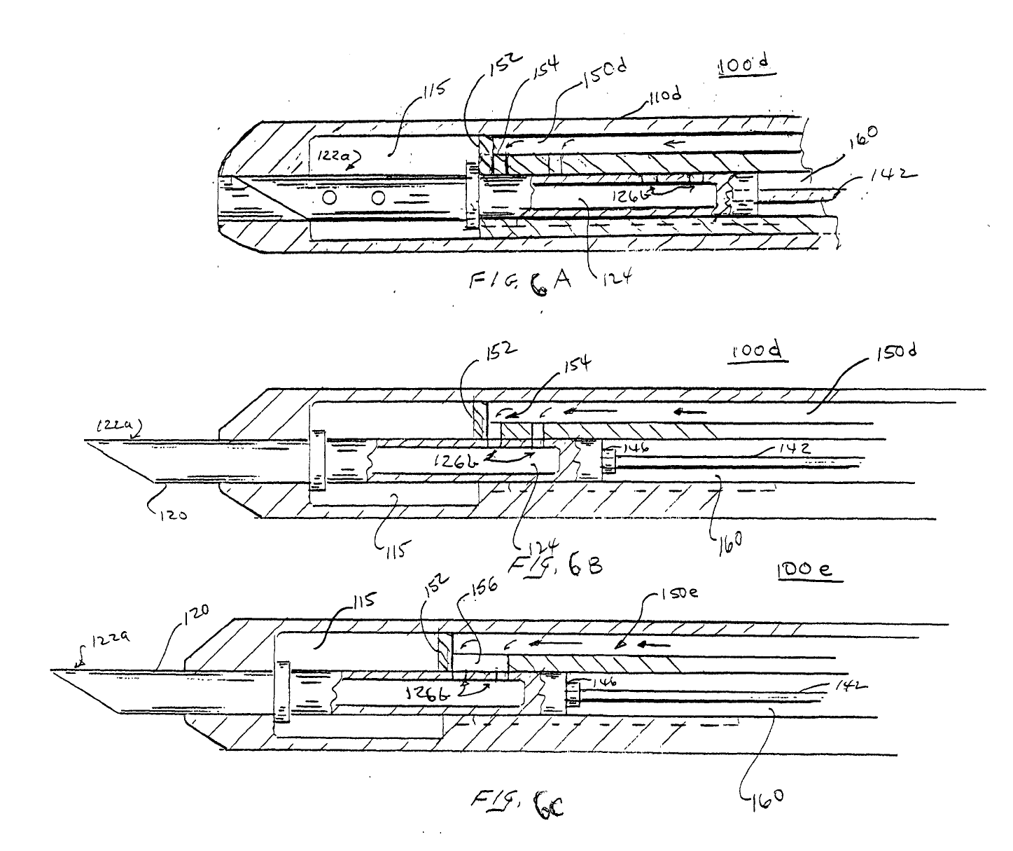

Referring now to FIGS. 4A and 4B there is shown section views of exemplary catheters 110a and 110b illustrating alternative fluid delivery lumens 150a and 150b and catheter passages 160a and 160b. There is shown in FIG. 4A a fluid delivery lumen 150a that is generally crescent shaped and in FIG. 4B, there is shown a fluid delivery lumen 150b that is in the form of a double-D or half-circle configuration. The foregoing is illustrative and shall not be considered as limiting the lumens/passages to the illustrated embodiments. It also is within the scope of the present invention for the fluid delivery lumen to be made up of lumens having different geometrical shapes. For example, the portion of the delivery lumen extending from the proximal end 114 of the catheter to about the location where the fluid is coupled to the delivery tip 120 can be configured as generally circular in cross-section, such as that shown in FIG. 2, and then changed over to the half circle or crescent shape at about the location where the fluid is being coupled to the through apertures 126b in the delivery tip.

Now referring to FIGS. 5A and 5B, there is shown a delivery apparatus 100c according to the present invention in which the catheter 110c includes a curved portion 117 such that a long axis of a catheter first portion 119a including the distal end 112 of the catheter is at an angle with respect to a long axis of a second portion 119b of the catheter that includes the proximal end 114 of the catheter. In the illustrated embodiment, the catheter first and second portions 119a and 119b are substantially straight or linear in configuration, however, this shall not be considered as a limitation on the present invention. In a more particular embodiment, the length of the catheter first portion 119a is minimized so the curved portion 117 is disposed closer to the distal end 112 of the catheter.

As used herein, the term curved portion 117 of the catheter refers to a segment or section of the of the catheter which is preferably curved, e.g., in the absence of an external or internal force inducing a deformation, a portion of the catheter outer tube is non-linear. In preferred embodiments, the curved portions comprise a smoothly curved tube without discontinuities or sharp corners. In particular embodiments, the curvature is a two dimensional curve including curvatures described by an arc of a circle, or a portion of an oval, ellipse, hyperbolic or the like. Alternatively, the curvature is a three dimensional curve including curvatures described by a section of a helix.

The angle of deflection for the curved section 117 is defined as the angle formed by the long axes of the first and second portions 119a and 119b of the catheter. In a particular embodiment, the curved portion has an angle of deflection θ in the range of from about 0° to about 45°, more particularly an angle of deflection θ in the range of from about 10° to about 40° and more specifically an angle of deflection θ of about 15°, about 20°, about 25°, about 30°, about 35° or about 40°.

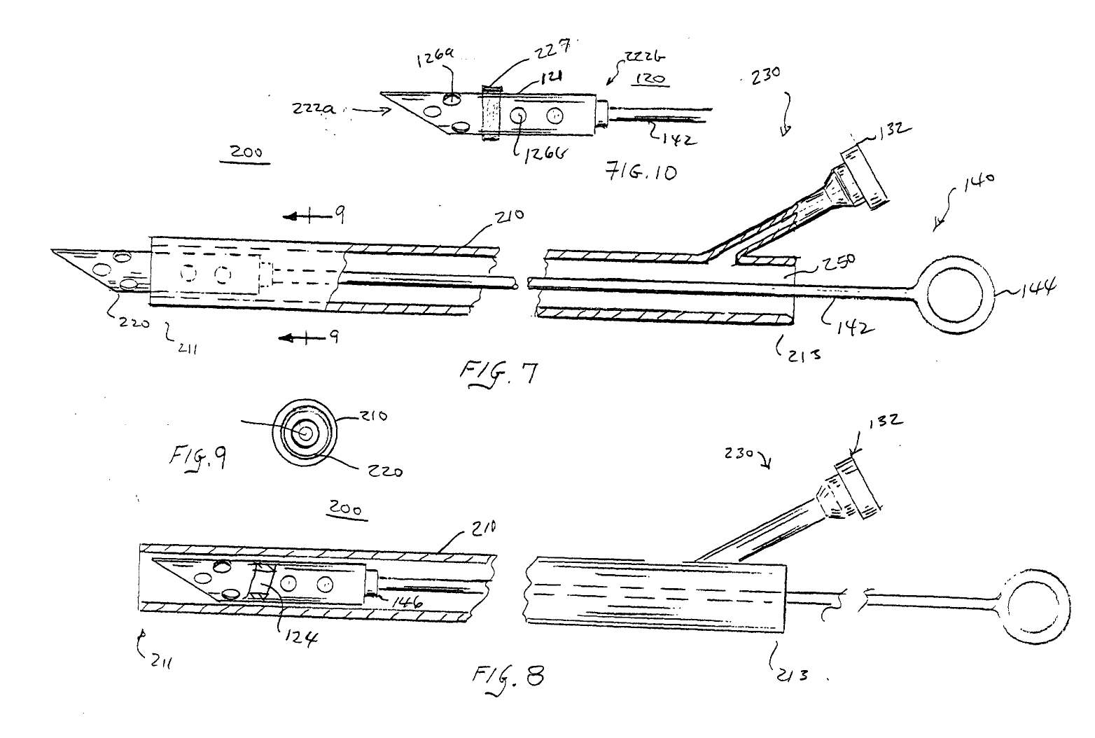

Referring now to FIGS. 6A and 6B there is shown various sectional side views of the distal end 112 of the catheter that illustrate alternate embodiment of a delivery apparatus 100d according to the present invention with the delivery tip retracted (FIG. 6A) and the delivery tip extended (FIG. 6B). Reference shall be made to the foregoing discussion regarding FIGS. 1-5 for common elements and features as well as details regarding related features not otherwise described or discussed below.

The delivery apparatus 100d according to this alternative embodiment includes a delivery tip 120, a catheter 110d and an actuation mechanism 140 that is coupled to the delivery tip. The catheter 110d of this embodiment further includes an inlet port 130 (FIG. 1). The inlet port 130 is fluidly coupled to a fluid delivery lumen 150d that is disposed within the catheter. Also disposed within the catheter 110d is a catheter passage 160 in which is located along a portion of the delivery tip 120 and a portion of the actuating mechanism 140, the connecting member 142.

In the illustrated embodiment, the fluid delivery lumen 150d includes a distal end-wall 152 that closes or seals off the distal end of the fluid delivery lumen and one or more through apertures 154 that extend radially through a sidewall of the lumen. These through apertures 154, are more particularly configured and arranged so that they are proximal to the portion of the delivery tip 120 in the catheter passage 160. More particularly and as shown in FIG. 6B, the lumen through apertures 154 and the through apertures 126b at the proximal end 122b of the delivery tip are configured and arranged such that when the delivery tip 120 is in the second or extended position, the lumen through apertures and the delivery tip through apertures are generally aligned so as to fluidly couple the delivery lumen 150d with the compartment 124 of the delivery tip.

In further embodiments, the catheter passage 160 and the delivery tip slidably disposed therein are configured and arranged with an alignment mechanism that maintains the relative alignment between the lumen through apertures 154 and delivery tip through apertures 126b. For example, the passage is configured to include a depressed track or slot in an interior surface thereof that extends lengthwise and the delivery tip is configured with a corresponding element or longitudinally extending tab that is slidably received in the lengthwise slot. In this way, the tab as it rides in the slot guides the delivery tip along a predetermined path and generally prevents lateral or rotational motion of the delivery tip.

Referring now to FIGS. 6A, 6B and 6C there is shown a sectional side view of the distal end 112 of the catheter that illustrate an alternate embodiment of a delivery apparatus 100d according to the present invention with the delivery tip retracted. Reference shall be made to the foregoing discussion regarding the preceding description of the embodiments for common elements and features as well as details regarding related features not otherwise described or discussed below.

The delivery apparatus 100d of FIGS. 6A-6C according to this alternative embodiment includes a delivery tip 120, a catheter 110d and an actuation mechanism 140 that is coupled to the delivery tip. The catheter 110d further includes an inlet port 130 (FIG. 1) which inlet port is fluidly coupled to a fluid delivery lumen 150d that is disposed within the catheter. Also disposed within the catheter 110d is a catheter passage 160 in which is located a portion of the delivery tip 120 and a portion of the actuating mechanism 140, the connecting member 142.

In the illustrated embodiment, the fluid delivery lumen 150d includes a distal end-wall 152 that closes or seals off the distal end of the fluid delivery lumen and a slotted through aperture 156 that extends circumferentially about a part of the lumen that is proximal to the portion of the delivery tip 120 in the catheter passage 160 and also extends radially through a sidewall of the lumen. More particularly, the slotted through aperture 156 and the through apertures 126b at the proximal end 122b of the delivery tip are configured and arranged such that when the delivery tip 120 is in the second or extended position. The slotted through aperture 156 and the delivery tip through apertures 126b are also generally aligned so as to fluidly couple the delivery lumen 150d with the compartment 124 of the delivery tip. The slotted configuration provides a mechanism to fluidly couple the various through apertures 126b and 156 while minimizing the lateral and circumferential positional accuracy required to align the delivery tip through apertures 126b.

In further embodiments, the catheter passage 160 and the delivery tip portion slidably disposed therein are configured and arranged with an alignment mechanism that maintains the relative alignment between the lumen slotted through aperture 156 and through apertures 126b of the delivery tip. For example, the passage is configured to include a depressed track or slot in an interior surface thereof that extends lengthwise and the delivery tip 120 is configured with a corresponding element or longitudinally extending tab that is slidably received in the lengthwise slot. In this way, the tab as it rides in the slot guides the delivery tip 120 along a predetermined path and generally prevents motion of the delivery tip in other directions.

The catheters 110a-e of the present invention can have any of a number of cross-sectional geometries as is known to those skilled in the art, including circular, oval, double-d, elliptical and polygonal cross-sections. In more particular embodiments, the catheters have a generally circular or oval cross section between about 5-8 french.

Also, the catheters 110 of the present invention as well as the components disposed within the catheter that can come into contact with the fluid being injected into targeted tissue of a body or patient are manufactured from any of a number of sterilizable, biocompatible, corrosion resistant materials known to those skilled in the art. Such biocompatible materials also yield a catheter that is sufficiently flexible to allow the catheter to be localized through a patient's vasculature to a specific target such as the interior of the heart, yet provide sufficient rigidity so that the catheter is resistant to collapse or kinking during such localization. In particular embodiments, such materials may include stainless steel, titanium, ceramics such as alumina, biocompatible plastics such as biocompatible-polycarbonates, polyethylene, polypropylene, poly(tetrafluoroethylene), i.e., Teflon, biocompatible polyesters, biocompatible polyamides, other biocompatible polymers and mixtures or blends thereof as well as various commercially available coatings thereon.

The use of the delivery apparatus according to an aspect of the present invention as well as methods for injecting a fluid or therapeutic agent into a specified tissue using such forms of the delivery apparatus described hereinabove can be best understood from the following discussion and with reference to FIGS. 1-6. It should be recognized that the catheters 110 comprising any of the delivery apparatuses of the present invention herein described are compatible with standard surgical insertion procedures for inserting a catheter into a vein or artery of a patient. The user, operator or medical personnel removes the delivery apparatus 100 from the sterile packaging and prepares the delivery apparatus for insertion into the patient's body such as through a patient's vasculature. In specific embodiments, such preparation includes manipulating the actuation member 144 so as to extend or retract the delivery tip 120 relative to the distal end 112 of the catheter. The delivery tip 120 is typically retracted so as to minimize incidental damage to the vasculature or other tissues while localizing the catheter distal end 112 proximal the site of the target tissue.

After the delivery apparatus 100 is prepared, the distal end 112 of the catheter is localized or located proximate the site of the target tissue. In particular applications suitable for use of the delivery apparatuses of the present invention, the distal end 112 of the catheter is localized through the vasculature to the interior of the heart. Typically, for cardiac therapies a catheter is inserted into an artery, usually a femoral artery in a leg or a brachial or radial artery in an arm. Frequently, such localizing also includes performing a diagnostic technique such as fluoroscopy, ultrasound IVUS or the like to facilitate localization of the distal end of the catheter at the target tissue site.

The type of tissue to which a therapeutic agent is to be delivered is not particularly limited. Organs and tissues, including heart, lung, kidney and skeletal muscles, which are accessible via the vasculature are preferred, e.g., organs and tissues which are supported by blood vessels having sufficiently large lumens for a catheter of the invention to pass through. A particularly preferred target organ is the heart including the aorta, left and right ventricles, interventricular septum and other regions thereof. Injection of a fluid or therapeutic agent into a patient's heart using a drug delivery catheter of the present invention is a particularly preferred application of the present invention.

After the distal end 112 of the catheter is localized to the target tissue, the user, operator or medical personnel (e.g., surgeon) manipulates/actuates the actuation member 144 so as to extend the delivery tip outwardly from the distal end of the catheter and so that the distal end 122a of the delivery tip including the needle point and the through apertures 126a penetrates or pierces the targeted tissue. When the delivery tip 120 is fully extended, the through apertures 126b at the proximal end 122b of the delivery tip are located within the distal end 112 of the catheter so as to be fluidly coupled with the catheter chamber 115 (FIG. 1), the lumen through aperture 154 (FIG. 6A) or the slotted through aperture 156 (FIG. 6C). In this way, the through apertures disposed within the targeted tissue are fluidly coupled to the catheter chamber 115, lumen through aperture 154 or slotted through aperture 156 via the compartment 124 of the delivery tip and the through apertures 126b at the proximal end of the delivery tip.

The user, operator or medical personnel (e.g., surgeon) then inject a fluid or therapeutic agent into the penetrated tissue via the through apertures 126a of the delivery tip disposed within the targeted tissue. This injection step includes attaching a syringe or other device to the fluid delivery adapter 132 and actuating such a syringe or other device so that the fluid or therapeutic agent is introduced into the fluid delivery lumen 150 and thence to the targeted tissues via the delivery tip.

If an additional fluid, drug or therapeutic agent is to be injected into new target tissue, the user/operator/medical personnel (e.g., surgeon) again manipulates the actuation member 144 so as to retract the delivery tip 120 back into the distal end 112 of the catheter. The user/operator/medical personnel localize the distal end 112 of the catheter at the site of the new target tissue and repeats the above described process of extending the delivery tip, penetrating the target tissue and injecting the fluid or therapeutic agent into the new target tissue. This process is repeated until there are no further tissues to be targeted.

At the completion of the foregoing process, the users/operator/medical personnel again retract the delivery tip 120 back into the distal end 112 of the catheter and withdraw the catheter 110 from the patient's body. The delivery tip 120 is preferably retracted into the distal end 112 of the catheter so as to minimize the risk of incidental damage to the vasculature or other tissues of the body during the removal of the catheter 110.

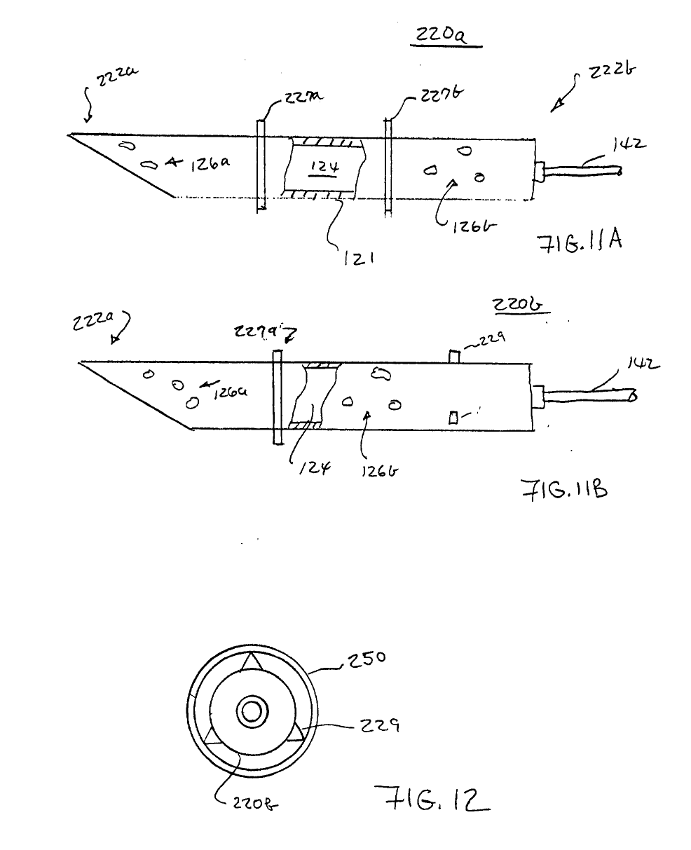

Now referring to FIGS. 7-10 there are shown various views of a delivery apparatus 200 or delivery system according to another aspect of the present invention. This embodiment of the delivery apparatus includes a catheter 210, a delivery tip 220 and an actuating mechanism 140 being coupled to the delivery tip. The catheter 210 further includes an inlet port 230 at or near a proximal end 213 thereof. The inlet port 230 is fluidly coupled to a fluid delivery lumen 250 that is disposed within the catheter. Also disposed within the fluid delivery lumen is a portion of the delivery tip 220 and a portion of the actuating mechanism 140 as well as a portion of the connecting member 142.

As shown in FIG. 10, the delivery tip 220 includes a tubular member 121 that is sealed at both a distal end 222a and a proximal end 222b thereof so as to form a substantially closed interior compartment or chamber 124 therein that generally extends lengthwise along a lumen of the catheter. The interior compartment 124 is generally defined by the interior surfaces of the tubular member 121 and the structures sealing each of the distal and proximal ends 222a and 222b. Reference shall be made to the foregoing discussion regarding the delivery tip 120 as shown in FIGS. 1-3 for details of common features not otherwise described below.

The distal end 222a of the delivery tip is configured and arranged so as to form a structure, such as a needlepoint, that can penetrate the tissue of the targeted region. In particular embodiments, the distal end 222a of the delivery tip is configured and arranged so as create an incision wound upon tissue penetration. In more specific embodiments, the distal end 222a of the delivery tip includes at least one cutting edge to facilitate formation of the incision, more particularly the delivery tip distal end is configured so as to form a structure having a beveled end with one or more cutting edges.

The proximal end 222b of the delivery tip is configured and arranged to provide a structure or structural member to which an end of the connecting member 142 of the actuating mechanism 140 can be operably secured thereto using any of a number of techniques known to those skilled in the art appropriate for the materials comprising the delivery tip proximal end and the actuating mechanism connecting member. In general, the proximal end 222b is configured and arranged to withstand the structural loading imposed thereon by the actuating mechanism 140 as the actuating mechanism causes the delivery tip 220 to be moved back and forth between the first and second positions.

The delivery tip also includes a raised area 227 that extends outwardly from the outer surface of the tubular member 121 and about the circumference of the tubular member. Additionally, the raised area 227 is disposed between the through apertures 126a at the distal end 222a of the delivery tip and the through apertures 126b at the proximal end 126b of the delivery tip. More particularly, the raised area 227 is located a sufficient distance back from the distal end 222a of the delivery tip so that when the delivery tip is extended from the distal end of the catheter, the raised area remains within the fluid delivery lumen 250. In addition, the raised area 227 is configured and arranged so as to establish a sliding fit between the raised region and the inner diameter of the fluid delivery lumen so as to form a fluid tight seal when the delivery tip is in either the retracted or extended positions. It is intended that the delivery tip may be formed of various lengths depending on the intended use and delivery location.

The fluid delivery lumen 250 is configured and arranged so that it generally extends between the distal and proximal ends 211, 213 of the catheter 210 and so a long axis of the fluid delivery lumen is generally, substantially parallel to a long axis of the catheter. The cross-sectional shape and size of the fluid delivery lumen 250 is defined so as to prevent or minimize damage to the one or more therapeutic agent(s) present in the fluid to be delivered via the fluid delivery lumen to the delivery tip 220.

As also indicated above, a portion of the actuating mechanism 140 and the connecting member 142 are disposed with the fluid delivery lumen 260 and extend from the proximal end 114 of the catheter to the proximal end 222b of the delivery device. As such, the connecting member 142 is configured and arranged so as to prevent or minimize damage to the one or more therapeutic agent(s) present in the fluid to be delivered via the fluid delivery lumen 250 to the delivery tip 220. In a more particular embodiment, the connecting member 142 is configured and arranged so has to have a cross-sectional area that is less than about 75% of the cross-sectional area of the fluid delivery lumen 250, more particularly the connecting member 142 is configured and arranged so as to have a cross-sectional area that is less than about 60%, 50%, 40% or 30% of the cross-sectional area of the lumen.

Also, at least a portion of the fluid delivery lumen is sized and configured so that a portion of the delivery tip is slidably disposed within the fluid delivery lumen. In addition, the fluid delivery lumen 250 is sized so that the connecting member 142 can move in the intended fashion, for example linearly, within the passage so as to shift the delivery tip 220 between and into the first and second (i.e., retracted and extended) positions.

Although the fluid delivery lumen 250 is illustrated as having a generally constant cross-section this shall not be construed as a limitation. It is within the scope of the present invention for the fluid delivery lumen 250 to be configured and arranged so as to have any of a number of configurations, sizes and cross-sections and geometric shapes as is known to those skilled in the art and otherwise appropriate for the intended use. For example, the portion of the delivery lumen 250 that slidably receives the delivery tip 220 can be configured and arranged so as to complement the delivery tip and the other portion of the delivery lumen housing the connecting member 142 can be configured and arranged so as to complement the connecting member.

As indicated above, in the illustrated embodiment the catheter 210 further includes an inlet port 230 at or near a proximal end 114 thereof. This inlet port 230 is fluidly coupled to the fluid delivery lumen 250 and an end of the inlet port is secured to a fluid delivery adapter 132. In more particular embodiments, the fluid delivery lumen 250 can have an open proximal end to which is coupled the fluid delivery adapter 132 or the proximal end of the fluid delivery lumen can be solid and the fluid delivery adapter is coupled to the fluid delivery lumen through a conduit in the sidewall of the fluid delivery lumen near the proximal end of the catheter 210.

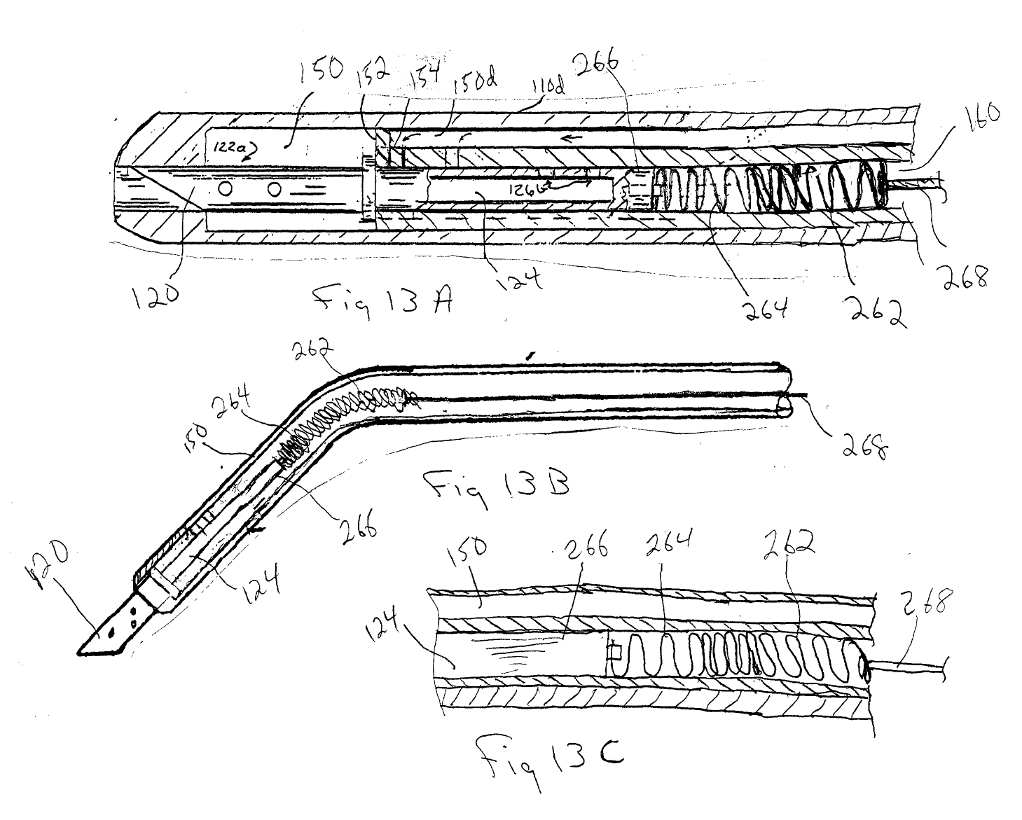

Referring now to FIGS. 11A and 11B there are shown alternative embodiments of delivery tips 220a and 220b for use in the delivery apparatus 200 according to another aspect of the present invention. In the embodiment illustrated in FIG. 11A, the delivery tip 220a includes a plurality of raised areas 227a and 227b that each extend outwardly from the outer surface of the tubular member 121 and about the circumference of the tubular member. Additionally, each of the raised areas 227a and 227b are disposed between the through apertures 126a at the distal end 222a of the delivery tip and the through apertures 126b at the proximal end 126b of the delivery tip. More particularly, the first raised area 227a is located a sufficient distance back from the distal end 222a of the delivery tip so that when the delivery tip is extended from the distal end of the catheter, the raised area remains within the fluid delivery lumen 250. In addition, each of the raised areas 227a and 227b are configured and arranged so as to establish a sliding fit between the raised region and the inner diameter of the fluid delivery lumen 250 so as to essentially form two fluid tight seals when the delivery tip is in either the retracted or extended positions. The second raised area 227b also is spaced from the first raised area 227a so as to generally maintain a long axis of the delivery tip parallel to a long axis of the catheter. In the alternative embodiment illustrated in FIG. 11B, the delivery tip 220b includes a first raised area 227a and a plurality of raised tabs 229 that each extend outwardly from the outer surface of the tubular member 121 and about the circumference of the tubular member. The first raised area 227a is disposed between the through apertures 126a at the distal end 222a of the delivery tip and the through apertures 126b at the delivery tip proximal end 126b. More particularly, the first raised area 227a is located a sufficient distance back from the distal end 222a of the delivery tip so that when the delivery tip is extended from the catheter distal end, the raised area remains within the fluid delivery lumen 250. In addition, the first raised area 227a is configured and arranged so as to establish a sliding fit between the raised region and the inner diameter of the fluid delivery lumen so as to essentially form a fluid seal when the delivery tip is in either the retracted or extended positions.

The plurality of tabs 229 are disposed in one of between the through apertures 126a at the delivery tip distal end 222a and the through apertures 126b at the proximal end 126b of the delivery tip (like that shown in FIG. 11A) or is disposed between the through apertures 126a at the proximal end 126b of the delivery tip and the proximal end 126b of the delivery tip (like that shown in FIG. 11B). In addition, each tab 229 is configured and arranged so as to establish a sliding fit between the tab and the inner diameter of the fluid delivery lumen 250. Because the tabs 229 are spaced from each other circumferentially, the fluid or therapeutic agent flows between the tabs to reach the delivery tip proximal end through apertures 126b. In an alternative embodiment, the tabs also are spaced from each other longitudinally.

The tabs 229 also are each spaced from the first raised area 227a so as to generally maintain a long axis of the delivery tip parallel to a long axis of the catheter. Although three tabs 229 are illustrated, this is not a limitation as it is within the skill of those knowledgeable in the art to configure the delivery apparatus with a plurality or more of such tabs. It also is within the scope of the present invention to configure the inner surface of the fluid delivery lumen 260 so as to include one or more depressed tracks or slots that extend lengthwise and to configure one or more of the tabs 229 so as to be slidably received within the lengthwise slot. In this way, as each tab 229 rides in the corresponding slot it guides the delivery tip along a predetermined path and generally prevents motion of the delivery tip in other directions.

Referring now to FIGS. 13A, 13B and 13C there is shown a sectional side view of the distal end 112 of the catheter that illustrate an alternate embodiment of a delivery apparatus 100 according to the present invention. Reference shall be made to the foregoing discussion regarding the preceding description of the embodiments for common elements and features as well as details regarding related features not otherwise described or discussed below.

The delivery apparatus 100 of FIGS. 13A-13C according to this alternative embodiment includes a delivery tip 120, a catheter 110 and an actuation mechanism 140 that is coupled to the delivery tip. The catheter 110 further includes an inlet port 130 (FIG. 1) which inlet port is fluidly coupled to a fluid delivery lumen 150 that is disposed within the catheter. Also disposed within the catheter 110 is a catheter passage 160 in which is located a portion of the delivery tip 120 and a portion of the actuating mechanism 140, the connecting member 142. In this embodiment, a plurality of springs, 262 and 264, are illustrated to provide longitudinal support for the distal portion of the catheter 110 as well as to provide a mechanism for the controlled bending of the distal portion of the catheter. The springs 262 and 264 also contribute to the ability of this device to advance or retract the delivery tip as desired. As referred to herein, the springs are oriented as interdigitated, spring within a spring or counterrotationally with respect to each other.

The actuation mechanism 140 of this embodiment preferably includes a modified connecting member 266 coupled to the proximal end 122b of the delivery tip, the second flange 128b and a ring member or actuation member 144 as shown in the embodiments of FIGS. 1-12. The connecting member 266 is similar to the connecting member 142 of the prior described embodiments except that it also includes a pair of interdigitated spring members 262 and 264. As discussed above, the connecting member is preferably formed of flexible, substantially non-compressible materials which are resistant to kinking or deformation upon application of a compressive force and other objects that can be housed inside of the catheter passage 160 and through which a mechanical force may be transmitted through at least a segment thereof to the delivery tip 120. Additionally, the actuation mechanism of this embodiment functions similar to the embodiments described above with the additional feature of providing a mechanism to impart a controlled bending of the distal end portion of the catheter while maintaining the rigidity of the catheter. As shown in FIGS. 13A-13C, the proximal spring member 262 may extend nearly the entire lengthwise dimension of the catheter or is attached to the distal end portion of an elongate wire member 268 such as the connecting member described above as connecting member 142. In the embodiment shown in the drawings, the proximal end portion of the proximal spring member 262 is connected to the distal end portion of the elongate wire member 268. Preferably, this connection allows the rotational movement between these two components while providing a substantially rigid longitudinal connections such that the proximal spring member 262 is rotatable relative the wire member 268 while maintaining a relatively constant overall lengthwise dimension for the actuation mechanism. In the preferred form of this embodiment, the distal spring member 264 is fixedly connected to the delivery device proximal end 122b.

In one form of this embodiment, as shown in the drawings, the distal spring member 264 may have a slight bend that is accentuated when the proximal spring member 262 is threaded relative to the distal spring member 264. As shown, the coils of each of the springs are spaced apart a sufficient distance to allow the coils of the opposite spring to fit within the space between the coils of the other spring member while one of the spring members is allowed to rotate relative to the other of the spring members. For example, as the connecting member 266 is moved distally relative to the body of the catheter, the proximal spring member 262 is pushed distally relative to the body of the catheter. The proximal spring member 262 also rotates relative to the wire member 268 and threads its self relative to the distal spring member 264 while preferably providing only minimal compression. As the proximal spring member 262 rotates relative to the distal spring member 264, the body of the catheter is also bent according the relative bend of the distal spring portion 264, if present. In this way, the user can control the amount of the bend of the distal end portion of the catheter while providing a rigid distal end of the catheter that will maintain its position against the desire tissue wall or membrane. As the proximal spring portion 264 also moves distally, the delivery tip 120 is also moved distally and the remainder of the catheter operates as described above with respect to FIGS. 1-12.

The use of the delivery apparatuses according to this aspect of the present invention as well as methods for injecting a fluid or therapeutic agent into a specified tissue using such a delivery apparatus described above can be best understood from the preceding discussions with respect to the delivery apparatuses disclosed in FIGS. 1-6. It should be recognized that the catheters 210 comprising any of the delivery apparatuses of the present invention herein described are compatible with standard surgical insertion procedures for inserting a catheter into a vein or artery of a patient. The user, operator or medical personnel removes the delivery apparatus 200 from the sterile packing and prepares the delivery apparatus for insertion into the patient's body such as through a patient's vasculature. In specific embodiments, such preparation includes manipulating the actuation member 144 so as to extend or retract the delivery tip 220 within the catheter distal end 212. The delivery tip 220 is typically retracted so as to minimize incidental damage to the vasculature or other tissues while localizing the catheter distal end 212 proximal the site of the target tissue. Although the delivery apparatus is shown as a multi lumen catheter, a single lumen catheter may also be used while a lumen is provided through the actuation mechanism to the delivery tip without departing form the intended operation and use of the present invention.

Although a preferred embodiment of the invention has been described using specific terms, such description is for illustrative purposes only, and it is to be understood that changes and variations may be made without departing from the spirit or scope of the following claims.

What is claimed is:

1. A system for delivering a fluid or therapeutic agent to target tissue of an organ such as a heart, said delivery system comprising:
a catheter having a distal end portion, a proximal end portion and a lumen extending therebetween;
a delivery device movably disposed within the catheter at the distal end portion thereof, the delivery device having a first end portion that is configured and arranged for penetrating the target tissue;
wherein the distal end portion of the catheter is configured and arranged so the delivery device is in one of a first position where the delivery device first end portion is disposed within the distal end portion of the catheter or in a second position where the first end portion of the delivery device is extended beyond the distal end portion of the catheter; and
wherein the distal end portion of the catheter is configured and arranged such that when the delivery device is in the second position a portion of the delivery device that is disposed within the distal end portion of the catheter is in fluid communication with the lumen, whereby fluid or therapeutic agent being communicated via the lumen is fluidly coupled to the first end portion of the delivery device and thence for delivery to the target tissue.

2. The delivery system of claim 1, further comprising an actuation member disposed within the catheter and being operably coupled to the delivery device, wherein the actuation member is configured and arranged so that the actuation member selectively moves the delivery device between, and into, the first position and the second position.

3. The delivery system of claim 2, wherein the distal end portion of the catheter is configured and arranged to include a mechanism to limit travel of the delivery device when the actuation member moves the delivery device from the first position to the second position.

4. The delivery system of claim 1, wherein the delivery device includes:
a passage that extends lengthwise within the delivery device;
a first aperture in the first end portion of the delivery device that is fluidly coupled to the passage; and
a second aperture in a portion of the delivery device that is disposed within the distal end portion of the catheter and the second aperture is fluidly coupled to the passage, whereby the fluid or therapeutic agent being communicated via the lumen is fluidly coupled to the first end portion of the delivery device via the first aperture, the passage and the second aperture.

5. The delivery system of claim 4, wherein
the distal end portion of the catheter is configured and arranged so as to include a passage in which the delivery device is moved from and between the first and second positions;
a portion of the passage includes a through aperture that is fluidly coupled to the lumen; and
when the delivery device is moved to the second position, the second aperture of the delivery device is fluidly coupled to the lumen via the through aperture.

6. The delivery system of claim 5, further comprising an actuation member disposed within the catheter separate from the lumen and being operably coupled to the delivery device, wherein the actuation member is configured and arranged so that the actuation member selectively moves the delivery device between, and into, the first position and the second position.

7. The delivery system of claim 4, wherein the second aperture of the delivery device is fluidly coupled to the lumen in either of the first or the second position thereof.

8. The delivery system of claim 7, further comprising an actuation member having a portion of which that is disposed within the lumen and is operably coupled to the delivery device, wherein the actuation member is configured and arranged so that the actuation member selectively moves the delivery device between, and into, the first position and the second position thereof.

9. The delivery system of claim 1, wherein the delivery device further includes a plurality of first apertures and a plurality of second apertures.

10. The delivery system of claim 1, wherein the distal end portion of the catheter is configured and arranged so that fluid or therapeutic agent within the lumen is not discharged from the delivery device when the delivery device is in the first position.

11. The delivery system of claim 4, wherein a portion of the passage is configured and arranged so as to form a fluid seal with the delivery device proximal of the first aperture of the delivery device so that fluid or therapeutic agent within the lumen is not discharged from the first aperture of the delivery device when the delivery device is in the first position.

12. The delivery system of claim 5, wherein the distal end portion of the catheter is configured arranged such that the second aperture of the delivery device is not fluidly coupled to the lumen when the delivery device is in the first position.

13. The delivery system of claim 1, wherein the catheter is configured and arranged such that the distal end portion is positionable within the heart of a patient and wherein the delivery device is configured and arranged to deliver the fluid or therapeutic agent to tissues of the patient's heart.

14. The delivery system of claim 1, further comprising an adapter located at or near the proximal end portion of the catheter and the adapter is fluidly coupled to the lumen, the adapter being configured and arranged so as to be capable of being fluidly coupled to the source of fluid or therapeutic agent that is to be communicated via the lumen.

15. The delivery system of claim 1 wherein the actuation member between the proximal end portion and distal end portion of the catheter and the actuation member includes a pair of coaxial spring members theralong.

16. The delivery system of claim 15 wherein the spring members are movable with respect to each other in response to movement of the actuation member.

17. A system for delivering a fluid or therapeutic agent to target tissue of a organ such as a heart from inside thereof, said delivery system comprising:
a catheter having a distal end portion, a proximal end portion and a lumen extending therebetween;
a delivery device movably disposed within the catheter at the distal end portion thereof;
an actuation member disposed within the catheter and being operably coupled to the delivery device;
wherein the delivery device includes a first end portion that is configured and arranged for penetrating the target tissue and a device passage that extends lengthwise within the delivery device,
a first aperture is located in the first end portion of the delivery device and is fluidly coupled to the device passage, and a second aperture is located in the portion of the delivery device that is disposed within the distal end portion of the catheter and is fluidly coupled to the device passage;
wherein the distal end portion of the catheter is configured and arranged so the delivery device is movable between a first position where the first end portion of the delivery device is disposed within the distal end portion of the catheter and a second position where the first end of the delivery device is extended beyond the distal end portion of the catheter;
wherein the distal end portion of the catheter is configured and arranged such that when the delivery device is in the second position the delivery device second aperture is put into fluid communication with the lumen, whereby the fluid or therapeutic agent being communicated via the lumen is fluidly coupled to the delivery device first end for delivery to the target tissue via the first aperture, the device passage and the second aperture; and
wherein the actuation member is configured and arranged relative to the distal end portion of the catheter so that the actuation member selectively moves the delivery device between, and into, the first position and the second position.

18. The delivery system of claim 17, wherein
the distal end portion of the catheter is configured and arranged so as to include a passage in the distal end portion of the catheter in which the delivery device is moved from and between the first and second positions;
a portion of the passage of the distal end portion of the catheter includes a through aperture that is fluidly coupled to the lumen; and
when the delivery device is moved to the second position, the second aperture of the delivery device is fluidly coupled to the lumen via the through aperture.

19. The delivery system of claim 17, wherein the second aperture of the delivery device is fluidly coupled to the lumen in either of the first or the second position of the delivery device.

20. The delivery system of claim 17, wherein the delivery device further includes one of a plurality of first apertures and one of a plurality of second apertures.

21. The delivery system of claim 17, wherein the distal end portion of the catheter is configured arranged such that the second aperture of the delivery device is not fluidly coupled to the lumen when the delivery device is in the first position.

22. The delivery system of claim 17, wherein the catheter is configured and arranged such that the distal end thereof is positionable within the heart of a patient and wherein the delivery device is configured and arranged with a precurved portion thereof to deliver the fluid or therapeutic agent to tissues of the patient's heart.

23. The delivery system of claim 17, further including an adapter located at or near the proximal end of the catheter and the adapter is fluidly coupled to the lumen and is configured and arranged so as to be fluidly coupled to the source of fluid or therapeutic agent that is to be communicated via the lumen to the patient.

24. The delivery system of claim 17 wherein the actuation member includes a pair of interdigitated spring members.

25. The delivery system of claim 24 wherein the spring members are movable with respect to each other in response to movement of the actuation member.

26. A method of injecting a fluid, therapeutic agent or drug into a heart of a patient, the method comprising:
providing a delivery system including a catheter having a distal end portion, a proximal end portion and a lumen extending therebetween;
a delivery device movably disposed within the catheter at the distal end portion thereof;
an actuation member disposed within the catheter and being operably coupled to the delivery device;
wherein the delivery device includes a first end portion that is configured and arranged for penetrating the target tissue and a device passage that extends lengthwise within the delivery device,
a first aperture is located in the first end portion of the delivery device and is fluidly coupled to the device passage, and a second aperture is located in the portion of the delivery device that is disposed within the distal end portion of the catheter and is fluidly coupled to the device passage;
wherein the distal end portion of the catheter is configured and arranged so the delivery device is movable between a first position where the first end portion of the delivery device is disposed within the distal end portion of the catheter and a second position where the first end of the delivery device is extended beyond the distal end portion of the catheter;
wherein the distal end portion of the catheter is configured and arranged such that when the delivery device is in the second position the delivery device second aperture is put into fluid communication with the lumen, whereby the fluid or therapeutic agent being communicated via the lumen is fluidly coupled to the delivery device first end for delivery to the target tissue via the first aperture, the device passage and the second aperture; and
wherein the actuation member is configured and arranged relative to the distal end portion of the catheter so that the actuation member selectively moves the delivery device between, and into, the first position and the second position;
inserting the distal end portion of the delivery system catheter into vasculature of the patient;
localizing the distal end portion of the catheter to the interior of the heart;
penetrating the targeted tissue with the first end portion of the delivery device; and
injecting a fluid or therapeutic agent into the heart tissue by discharging the fluid or therapeutic agent from the first end portion of the delivery device.

27. The method of claim 26 wherein the actuation member is provided with a pair of spring members and wherein movement of the actuation member toward the distal end portion of the delivery system causes the distal end portion of the catheter to assume a precurved orientation.

28. A method of injecting a fluid, therapeutic agent or drug into targeted tissue of a patient, the method comprising:
providing a delivery system including a catheter having a distal end portion, a proximal end portion and a lumen extending therebetween;
a delivery device movably disposed within the catheter at the distal end portion thereof, the delivery device having a first end portion that is configured and arranged for penetrating the target tissue;
wherein the distal end portion of the catheter is configured and arranged so the delivery device is in one of a first position where the delivery device first end portion is disposed within the distal end portion of the catheter or in a second position where the first end portion of the delivery device is extended beyond the distal end portion of the catheter; and
wherein the distal end portion of the catheter is configured and arranged such that when the delivery device is in the second position a portion of the delivery device that is disposed within the distal end portion of the catheter is in fluid communication with the lumen, whereby fluid or therapeutic agent being communicated via the lumen is fluidly coupled to the first end portion of the delivery device and thence for delivery to the target tissue;
inserting the distal end portion of the delivery system catheter into vasculature of the patient;
localizing the distal end portion of the delivery system catheter to the targeted tissue of the patient;
penetrating the targeted tissue with the first end portion delivery device; and
injecting a fluid or therapeutic agent into the targeted tissue by discharging the fluid or therapeutic agent from the first end portion of the delivery device.
